(19) United States
(12) Patent Application Publication
(10) Pub. No.: US 2025/0221887 A1
(43) Pub. Date: Jul. 10, 2025

(54) SLEEP IMPROVEMENT DEVICE USING VIBRATORY STIMULATION, AND SLEEP IMPROVEMENT METHOD USING SAME

(71) Applicant: BRLAB INC., Seoul (KR)

(72) Inventors: Hyun Bin KWON, Seoul (KR); Jong Min Lee, Seoul (KR); Il Song Lee, Seoul (KR); Hui Nam Yoon, Seoul (KR)

(21) Appl. No.: 19/093,726

(22) Filed: Mar. 28, 2025

Related U.S. Application Data
(63) Continuation of application No. PCT/KR2023/014770, filed on Sep. 26, 2023.

(30) Foreign Application Priority Data
Sep. 30, 2022 (KR) 10-2022-0124872
Dec. 22, 2022 (KR) 10-2022-0182003
Dec. 22, 2022 (KR) 10-2022-0182004
Apr. 10, 2023 (KR) 10-2023-0047029

(51) Int. Cl. A61H 23/02; A61B 5/00

(52) U.S. Cl. CPC A61H 23/0254; A61B 5/4806; A61B 2562/0247

(57) ABSTRACT

The present disclosure relates to an unrestrained sleep improvement device and a sleep improvement method using the same, and more particularly, to a sleep improvement device that senses biometric information items from a user on a bed or mattress, identifies a sleep state of the user from the sensed biometric information items, and generates and provides stimulation for inducing a change in the user's biometric signal according to the identified sleep state, and a sleep improvement method using the same.

### CROSS-REFERENCE TO RELATED APPLICATIONS

This application is a Continuation of Application No. PCT/KR2023/014770, filed on Sep. 26, 2023, which in turn claims the benefit of Korean Patent Applications No. 10-2022-0124872, filed on Sep. 30, 2022, No. 10-2022-0182003, filed on Dec. 22, 2022, No. 10-2022-0182004, filed on Dec. 22, 2022, and No. 10-2023-0047029, filed on Apr. 10, 2023. The entire disclosures of all these applications are hereby incorporated by reference.

### TECHNICAL FIELD

The present disclosure relates to a sleep improvement device and a sleep improvement method using the same, and more particularly, to a device and method of applying vibratory stimulation to a user so as to induce a sleep improvement effect.

### BACKGROUND ART

Various attempts are being made to induce users to get a deep sleep. There is a popular way to create a good environment for falling asleep by playing quiet music or generating white noise around the users when they sleep. However, it is difficult to change a person's sleep state in a meaningful way by relying only on external noise, and in particular, there are limitations in inducing a deep sleep state while considering a user's health.

The present disclosure is conceived during a research and development process to effectively allow a user to enter a deep sleep state, and in particular, a main object of the present disclosure is to allow a user to enter a deep sleep state by applying appropriate vibratory stimulation to the user.

### DISCLOSURE OF INVENTION

### Technical Problem

An aspect of the present disclosure is to induce a user lying on a mattress to enter a deep sleep state by applying vibratory stimulation.

In addition, an aspect of the present disclosure is to allow appropriate vibratory stimulation to be applied to a user not only on a flat mattress but also on a folded mattress.

In addition, an aspect of the present disclosure is not only to simply provide vibratory stimulation, but also to control the parameters of the vibratory stimulation to be applied by acquiring and analyzing a biometric signal of a user lying on a mattress.

Meanwhile, technical problems of the present disclosure are not limited to the above-mentioned problems, and other technical problems which are not mentioned herein will be clearly understood by those skilled in the art from the description below.

### Technical Solution

The present disclosure is provided to solve the foregoing problems, and a sleep improvement device according to the present disclosure may include a plurality of layers; and a vibration unit provided in any one of the plurality of layers to output a vibration signal, wherein the vibration unit is provided so as to output a vibration signal toward an upper side of the mattress.

Furthermore, the sleep improvement device may further include a vibration sensing unit provided in any layer to acquire a vibration signal from the sleep improvement device or a user; and a pressure sensing unit provided in an arbitrary layer to acquire a pressure signal according to the user's movement.

Furthermore, in the sleep improvement device, the vibration unit may include an actuator that generates vibration, wherein the actuator is in contact with a lower surface of a vibration plate, and the vibration plate is provided to be in contact with a lower surface of the mattress.

Furthermore, the sleep improvement device may further include a vibration transmitting body for amplifying or attenuating the vibration signal, the vibration transmitting body that is present between the vibration unit and an upper surface of the mattress.

Meanwhile, in a sleep improvement device according to another embodiment of the present disclosure, the sleep improvement device may include at least two split beds, wherein each split bed includes a plurality of layers; a vibration sensing unit provided in any layer among the plurality of layers to acquire a vibration signal from the split bed or a user's body in contact with an upper surface of the split bed; and a pressure sensing unit provided in an arbitrary layer to acquire a pressure signal according to the user's movement.

Furthermore, in the sleep improvement device, at least one of the plurality of split beds may further include a vibration unit that outputs a vibration signal.

Furthermore, in the sleep improvement device, the sleep improvement device may include a first split bed in contact with the user's back or stomach; and a second split bed in contact with the user's legs.

Furthermore, in the sleep improvement device, the first split bed may include a first-first vibration sensing unit and a first-first pressure sensing unit provided at a top of the first split bed; and a first-second vibration sensing unit and a first-second pressure sensing unit provided at a bottom of the first split bed.

Meanwhile, in a sleep improvement method using a sleep improvement device according to still another embodiment of the present disclosure, the sleep improvement device may include a plurality of layers; and a vibration unit provided in any one of the plurality of layers to output a vibration signal, the vibration unit being provided so as to output a vibration signal toward an upper surface of the mattress, and the sleep improvement method may include sensing a user's lying posture on a layer of the sleep improvement device; and controlling the vibration unit to output a vibration signal toward an upper surface of the mattress.

Meanwhile, in a sleep improvement method using a sleep improvement device according to yet still another embodiment of the present disclosure, the sleep improvement device may include at least two split beds, wherein each split bed includes a plurality of layers constituting each split bed; a vibration sensing unit provided in any layer among the plurality of layers to acquire a vibration signal from the split bed or a user's body in contact with an upper surface of the split bed; a pressure sensing unit provided in an arbitrary layer to acquire a pressure signal according to the user's movement; and a vibration unit included in at least one of the plurality of split beds to output a vibration signal, and the sleep improvement method may include sensing the presence of a user applying pressure to any one of the split beds of the sleep improvement device; and controlling the vibration unit to output a vibration signal toward an area to which pressure is applied by the user.

### Advantageous Effects

According to the present disclosure, vibratory stimulation may be used to effectively induce a user's sleep state.

In addition, according to the present disclosure, not only vibratory stimulation but also sound stimulation may be generated, thereby allowing more diverse combinations of stimulation transmission.

In addition, according to the present disclosure, there is an effect of efficiently transmitting stimulation according to the user's lying posture not only on a flat surface but also on a folded mattress.

In addition, according to the present disclosure, an appropriate stimulation program may be generated based on biometric signals collected from the user, thereby having an effect of allowing the user to enter a deep sleep state.

Meanwhile, the effects of the present disclosure may not be limited to the above-mentioned effects, and other technical effects which are not mentioned herein will be clearly understood by those skilled in the art from the description below.

### BRIEF DESCRIPTION OF THE DRAWINGS

FIG. 1 conceptually organizes the components of a sleep improvement device according to the present disclosure.

FIG. 2a, FIG. 2b and FIG. 3 show one embodiment of implementing a structure of a vibration unit.

FIG. 4 shows one embodiment of a vibration plate.

FIG. 5 shows a plurality of vibration units that can be provided on a layer.

FIG. 6 shows a plurality of vibration units provided on a folded mattress.

FIGS. 7 and 8 shows a configuration in which a plurality of sensing units are further provided on the mattress shown in FIG. 6.

### BEST MODE FOR CARRYING OUT THE INVENTION

The details of the objects and technical configurations of the present disclosure and operational effects thereof will be more clearly understood from the following detailed description based on the accompanying drawings appended hereto. Hereinafter, embodiments according to the present disclosure will be described in detail with reference to the accompanying drawings.

Embodiments disclosed herein should not be interpreted as limiting or used to limit the scope of the present disclosure. It is apparent for those skilled in the art that a description including embodiments herein has various applications. Therefore, any embodiments described in the detailed description of the present disclosure are illustrative for better understanding of the present disclosure and are not intended to limit the scope of the present disclosure to the embodiments.

Functional blocks illustrated in the drawings and described hereunder are only examples of possible implementations. In other implementations, other functional blocks may be used without departing from the concept and scope of the detailed description. Furthermore, one or more functional blocks of the present disclosure are illustrated as separate blocks, but one or more of the functional blocks of the present disclosure may be a combination of various hardware and software elements that execute the same function.

In addition, an expression that some elements are “included” is an expression of an “open type”, and the expression simply denotes that the corresponding elements are present, but should not be construed as excluding additional elements.

Moreover, in case where it is mentioned that one element is “connected” or “coupled” to the other element, it should be understood that one element may be directly connected to the other element, but another element may be present therebetween.

### Embodiment of Flat General Mattress

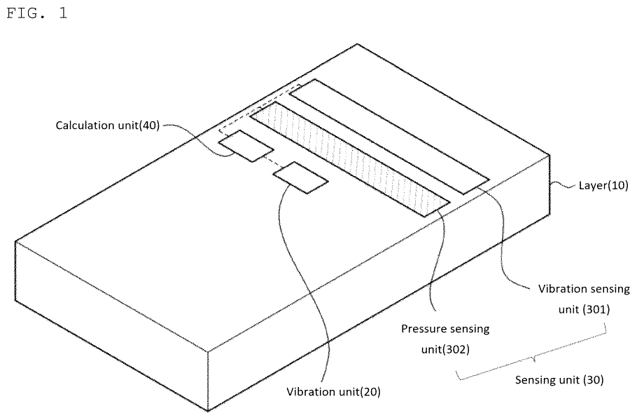

FIG. 1 shows a sleep improvement device according to a first embodiment of the present disclosure. Referring to FIG. 1, it can be seen that the sleep improvement device may largely include a layer 10, a vibration unit 20, a sensing unit 30, and a calculation unit 40, and the sensing unit 30 may be further divided into a vibration sensing unit 301 and a pressure sensing unit 302. Hereinafter, for an overall understanding of the sleep improvement device, a detailed description will be given for each component.

First, the layer 10 may be understood as a member having a receiving space in which the components to be described later can be arranged, and if a receiving space is provided, there is no limitation on the material or shape of the layer 10. The layer 10 may be, for example, a mattress, or may be any one of a plurality of surfaces constituting a mattress. Additionally, the layer 10 may be a mat that can be placed on a mattress, and may furthermore be a member made of wood or metal rather than cotton with a fiber material. In this manner, if the layer 10 has a predetermined receiving space, there is no limitation on the material or shape. However, in order to help understand the disclosure, in this detailed description, the explanation will be continued assuming that the layer 10 is a mattress.

The vibration unit 20 is configured to generate vibratory stimulation to be transmitted to a user lying on a mattress. The vibration unit 20 may be provided to output a vibration signal particularly toward an upper surface of the mattress, that is, a surface on which the user is lying. In addition, the vibration unit 20 may refer to converting an electrical signal into at least one of sound energy and vibration energy to output the converted energy. In other words, the vibration unit 20 may be configured to output sound and vibration simultaneously.

Vibratory stimulation may be used for a variety of purposes, such as inducing a change in the user's biometric signal or waking the user up according to alarm information.

The user's biometric signals may include various signals that can be sensed from the user, and a vibration signal, a pressure signals, and the like that can be sensed from the user lying on the mattress may be one type of biometric signal. The meaning of inducing a change in biometric signal may ultimately refer to inducing the user's sympathetic or parasympathetic nerves to be activated through simulation in a way that improves the user's sleep quality. More specifically, the user's parasympathetic nerves may be activated to a desired state to allow the user to get a good quality of sleep, and the user's sympathetic nerves may be activated to allow the user to be induced to wake up at an appropriate time or at a set time.

Alarm information refers to information items set by the user to output a vibration signal at a specific time, and the alarm information may include visual information regarding when to output the vibration signal, intensity information regarding a strength of the vibration signal, or duration information regarding how continuously to output the vibration signal. Additionally, the alarm information may include user identification information regarding the user lying on which side of the mattress to output the vibration signal. User identification information may refer to information for identifying a specific user, or may mean location information for indicating whether a vibration signal is to be output toward a user lying on the left or right side on the mattress.

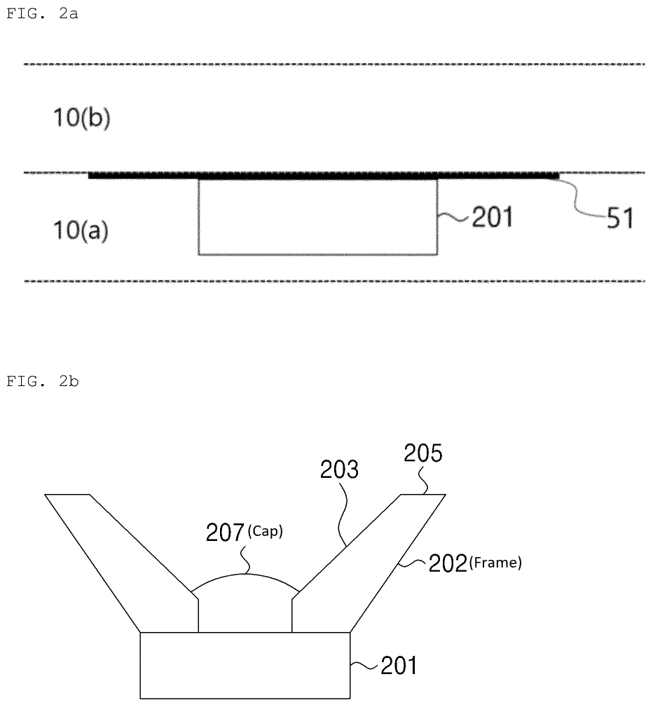

A more specific structure of the vibration unit 20 will be examined again in the description of FIG. 2, and herein, a description of the remaining components of the sleep improvement device will be continued below.

The sensing unit 30, which is provided on the layer 10, is configured to sense and acquire biometric information from a user lying on a mattress. As mentioned above, the sensing unit 30 may include a vibration sensing unit 301 and a pressure sensing unit 302, each of which, as can be seen from their names, may sense a vibration signal or a pressure signal that can be felt on the mattress.

The vibration sensing unit 301 may preferably be implemented with polyvinylidene fluoride (PVDF). The PVDF is a piezoelectric material that generates electricity when mechanical/physical stimulation is applied, and this property may be used to sense a vibration signal sensed on a mattress. The vibration sensing unit 301 may sense vibration to obtain at least one of the user's biometric information, particularly a ballistocardiogram, a respiratory state, or a movement state, or may sense vibration caused by vibratory stimulation or vibration signal output by the vibration unit 20 described above. In some cases, the vibration sensing unit 301 may be positioned relatively closer to an upper surface of the mattress to better sense biometric signals (ballistocardiogram, etc.) from the user, and may be positioned in the layer 10 relatively closer to the vibration unit 20 to better sense vibration from the vibration unit 20.

The pressure sensing unit 302 may preferably be implemented as an a force sensing resistor (FSR) array. The FSR is a component that converts a pressure value into a resistance value, and if a plurality of FSRs are provided in an array form, the user's posture may be estimated by referring to magnitudes of resistance values sensed on a mattress using the above property.

Lastly, the calculation unit 40 controls the components responsible for a function of the sleep improvement device, such as the vibration unit 20 and the sensing unit 30 described above. The calculation unit 40 may also be understood as a central processing unit. The central processing unit may also be referred to as a controller, a microcontroller, a microprocessor, a microcomputer, or the like. Furthermore, the central processing unit may be implemented by hardware or firmware, software, or a combination thereof, and configured to include an application specific integrated circuit (ASIC) or a digital signal processor (DSP), a digital signal processing device (DSPD), a programmable logic device (PLD), or a field programmable gate array (FPGA) when implemented using hardware, and configured with firmware or software to include a module, a procedure, a function or the like that performs the foregoing functions or operations when implemented using firmware or software. In addition, the calculation unit 30 may include a memory, and the memory may be implemented as Read Only Memory (ROM), Random Access Memory (RAM), Erasable Programmable Read Only Memory (EPROM), Electrically Erasable Programmable Read-Only Memory (EEPROM), flash memory, Static RAM (SRAM), a hard disk drive (HDD), a solid state drive (SSD) or the like.

FIGS. 2A and 2B show the detailed structure of the vibration unit 20.

First, referring to FIG. 2A, the vibration unit may be implemented with the actuator 201 in contact with a lower surface of a vibration plate 51. The actuator 201 is a component that converts an electrical signal into a vibration signal and outputs it, and may be said to be a component that actually generates vibration. There is no limitation on the type of the actuator 201 as long as it has a structure capable of generating vibration, but it may preferably be implemented by a motor capable of generating vibration, or a combination of a motor and a rotor. Referring to the drawing, the location of the actuator 201 may be a first layer 10(a), and the vibration plate 51 may be provided on a surface where the first layer 10(a) and the second layer 10(b) are in contact with each other so as to allow vibration generated from the actuator 201 to be spread by the vibration plate 51 and transmitted to the second layer 10 (b). In addition, the shape of the actuator 201 may be implemented in various ways, but may preferably have a cylindrical housing with components such as a motor built into the housing to directly generate vibration.

Next, FIG. 2B shows a structure of the vibration unit 20 according to another embodiment. Referring to the drawing, the vibration unit may include, as its main components, an actuator 201 that generates vibration, a transmission surface 203 that extends from the actuator 201 to transmits vibration, and an edge surface 205 in contact with a surface of a mattress (layer) that is to generate vibration to transmit vibration from the transmission surface 203 to the surface of the layer. In addition, the vibration unit 20 may further include a frame 202 for forming a cone-shaped structure and a cap 207 for protecting the actuator 201 from contamination by external substances.

The transmission surface 203 provided on a top of the actuator 201 is configured to transmit vibration generated by the actuator 201 to the other side, and the transmission surface 203 may be made of a material capable of transmitting a vibration signal, such as wood, metal, or silicone. Additionally, the transmission surface 203 may have a cone shape, in other words, a triangular pyramid or tripod shape. FIG. 2B shows a cross-sectional side view of the vibration unit 20, according to which the frame 202 and the transmission surface 203 may have a shape that widens upward from the actuator 201, and more precisely, may have a cone-shaped structure.

The edge surface 205 is a flat surface connecting an upper side of the transmission surface 203 and an upper side of the frame 202, wherein the edge surface 205 may be in contact with an inner side of the upper surface of the mattress to transmit a vibration signal from the actuator 201.

Meanwhile, a vibration transmitting body 250 may be additionally provided on a top of the actuator 201, more precisely, in a space between the vibration plate 51 and the actuator 201. There is no problem in directly transmitting a vibration signal to the layer (mattress) simply by placing the vibration plate 51 on the actuator 201, but the vibration transmitting body 250 for more effective transmission of the vibration signal or for changing the properties of the vibration signal may be further provided as in FIG. 3. As shown in FIG. 3, the vibration transmitting body 250 may be formed with the same area and shape as the area of the actuator 201 when viewed from above, and preferably, the actuator 201 and the vibration transmitting body 250 may be designed to be structurally connected to each other or structurally fitted together, thereby implementing the actuator 201 and the vibration transmitting body 250 to vibrate as a single mass. For example, the actuator 201 may be implemented to have a convex cross-section, and the vibration transmitting body 250 may be implemented to have a donut shape that can be fitted to the actuator 201. In addition, a ring-shaped component such as a rubber ring or bearing may be further provided between the actuator 201 and the vibration transmitting body 250 to increase or decrease the resistance of the surface where the actuator 201 and the vibration transmitting body 250 come into contact. A volume and area (an area in a plan view) of the vibration transmitting body 250 may have larger values than those of the actuator 201, and the volume or cross-sectional area may be determined depending on the vibration properties that the designer wants to implement. For example, when it is desired to transmit vibration only to a local area, the vibration transmitting body 250 may be designed to have an area smaller than or equal to that of the actuator 201 based on the plan view, and when it is desired to spread vibration to a wider area, an area of the vibration transmitting body 250 may be designed to have a larger value than that of the actuator 201. In addition, the vibration transmitting body 250 may be manufactured from the same material as the actuator 201, but may also be manufactured from a different material than the actuator 201. For example, the vibration transmitter 250 may be implemented with various materials such as wood, a solid, a polymer compound, or a fluid (e.g., a vibration transmitting body containing a fluid in a pouch made of a polymer compound).

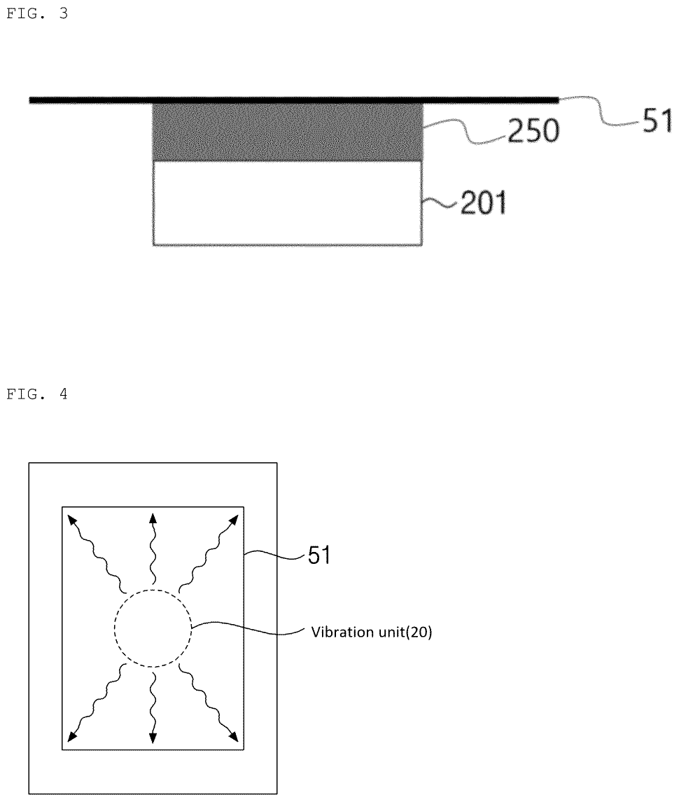

Next, the vibration plate 51 will be examined in more detail with reference to FIG. 4. The vibration plate 51 is provided to spread and transmit vibration generated by the vibration unit 20 over a relatively wide area. The vibration plate 51 may be made of a different type of material from the layer 10 or the actuator 201, and for example, wood, stone, metal, or polymer compounds may be used as materials for manufacturing the vibration plate 51.

Regarding a thickness of the vibration plate 51, the thickness may vary depending on the material, but preferably, the thickness may be set within a range of 1 mm to 20 mm. The vibration plate 51 may be covered with a separate protective surface made of a flexible material to prevent cracks or damage. For example, at least one of front and back surfaces of the vibration plate 51 made of stone may be covered with a protective surface of silicone material, thereby minimizing cracks or damage of the stone vibration plate 51. In addition, the protective surface may also improve a structural stability of the vibration plate 51 and the vibration unit 20 by improving an adhesive strength and inter-surface resistance with a surface of the layer 10b that comes into contact with the vibration plate 51.

An area of the vibration plate 51 may be determined according to the designer's intention, but may preferably be determined within a range above an area of the actuator 201 as viewed in a plan view and below an area of the mattress (layer) as viewed in a plan view. As needed, the vibration plate 51 may be formed so wide that its area occupies a significant portion of the mattress as shown in FIG. 4. In an embodiment where a significant portion of the mattress is covered with the vibration plate 51, the covered area may be about 50% to 80% of a total area of the mattress on a plane, and preferably, an outer portion of the mattress that is not easily in contact with the user's body is not covered by the vibration plate 51, thereby preventing the area of the vibration plate 51 from becoming excessively large.

On the other hand, although not specifically indicated in the drawing, the vibration plate 51 may not necessarily be made of only one material. For example, by placing a first vibration plate on the left side where the first user lies and a second vibration plate on the right side where the second user lies, two people lying on the mattress may feel different vibration sensations. Each vibration plate may preferably be installed in contact with a separate vibration unit 20, but in some cases, one vibration unit 20 may be implemented to selectively vibrate either one or both of the two vibration plates. In the former case, it may be implemented by allowing only the vibration plate that needs to transmit vibration to be selectively in contact with the vibration unit 20.

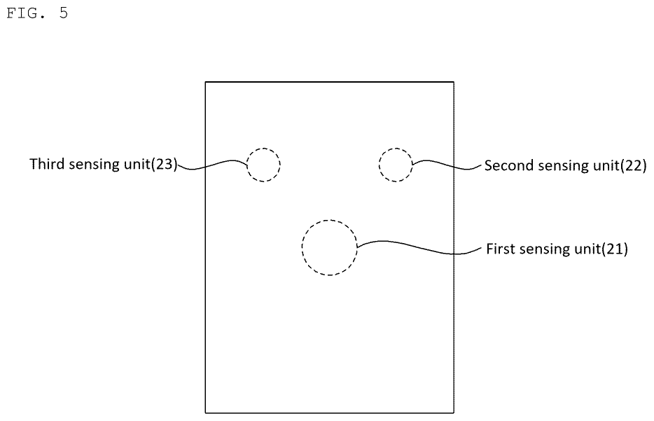

FIG. 5 shows a configuration in which a plurality of vibration units 21, 22, 23 are provided in a sleep improvement device according to the present disclosure, and referring to the drawing, a first vibration unit 21 may be provided at the center of the mattress, and a second vibration unit 22 and a third vibration unit 23 may be provided at upper right and upper left sides of the mattress, respectively, where the user's shoulder can touch. The second vibration unit 22 and the third vibration unit 23 may be implemented in the same manner as the structure of the vibration unit described above with reference to FIG. 2, but are not necessarily limited thereto. That is, the second vibration unit 22 and the third vibration unit 23 may have a structure that outputs a vibration signal in a different manner from the first vibration unit 21.

In addition, an operation of a plurality of vibration units may be individually controlled by a control command of the calculation unit 40, and in particular, the vibration unit to be operated to induce the user into a specific sleep state according to the biometric signal acquired from the user or to wake the user according to the user's alarm information, and a vibration intensity, a vibration cycle of the vibration unit may be controlled.

In the above, structures of sleep improvement devices that can utilize vibration signal output have been examined with reference to FIGS. 1 to 5.

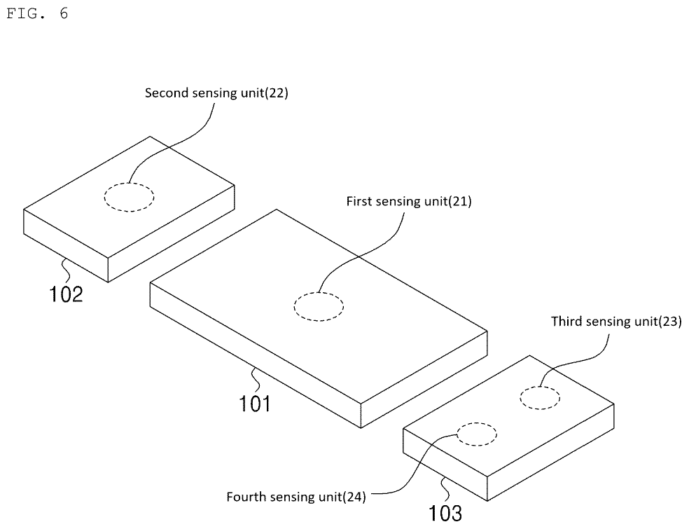

FIGS. 6 to 8 show a configuration in which vibration units and sensing units are provided on a folded mattress. A folded mattress refers to a mattress that is divided into a first split bed 101, a second split bed 102, and a third split bed 103 as shown in FIG. 6 so as to be folded according to a user's operation. If the sleep improvement device in FIGS. 1 to 5 described above is described based on the assumption of a flat, non-folded mattress, FIGS. 6 to 8 are shown based on the assumption of a mattress that can be folded in this manner.

In the case of a folded mattress, a surface area of the user's body in touch with an upper surface of the mattress may vary depending on how much each split bed is folded. For example, when the mattress is folded as in FIG. 6, the user's buttocks may be in the closest contact with the first split bed 101, the user's shoulder may be in the closest contact with the second split bed 102, and the user's calves may be in the closest contact with the third split bed 103. As in the present disclosure, when it is desired to improve the user's sleep state or transmit an alarm signal by using a vibration signal, it is necessary to output a vibration signal toward the user's body part that is in the closest contact with the folded mattress, and to this end, it is necessary to provide vibration units in a manner shown in FIG. 6.

Referring to FIG. 6, it can be seen that the first vibration unit 21 may be provided on the first split bed 101, the second vibration unit 22 may be provided on the second split bed 102, and the third vibration unit 23 and the fourth vibration unit 24 may be provided on the third split bed 103. The two vibration units on the third split bed 103 are intended to output vibration signals corresponding to the user's two legs (calves).

As described above, the operation of each vibration unit may be individually controlled by a calculation unit (not shown), and preferably, the operation may be individually controlled according to a degree of folding of each split bed, or according to the user's lying or sitting posture, which is analyzed based on biometric signals acquired from the user. For example, when the user's body part is not in close contact with the mattress surface due to a degree of folding of each split bed, or the body part is in contact with a surface of the mattress but the contact due to gravity is not at a preset level, there is no need to operate the vibration unit provided in the split bed, and conversely, only the vibration units in the split beds in which the contact is above the preset level need to be operated.

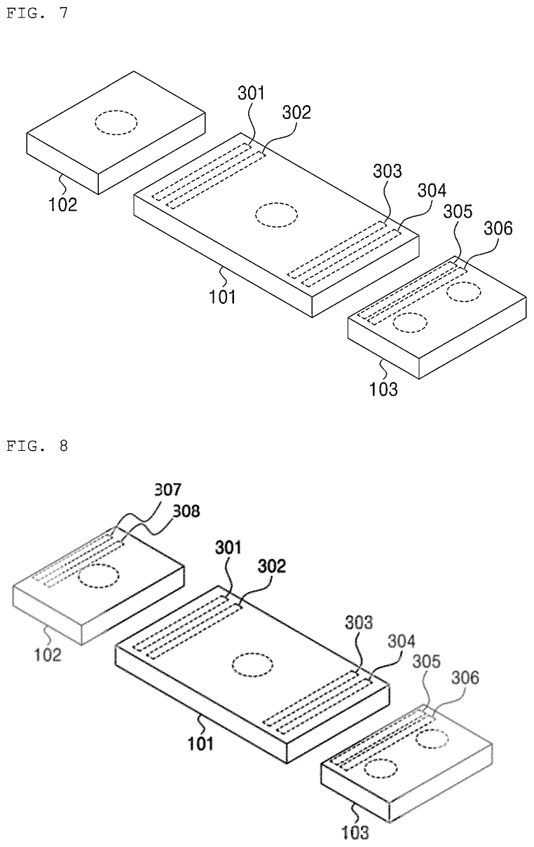

Meanwhile, in order to estimate a degree to which the user's body part is in close contact with the mattress or a posture in which the user is lying on the folded mattress, the components of the vibration sensing unit 301 and the pressure sensing unit 302 described above in FIG. 1 are further required, which may be provided as shown in FIG. 7 or 8.

Referring to FIG. 7 or 8, there is shown a configuration in which a first vibration sensing unit 301 and a first pressure sensing unit 302 are provided at an upper end of the first split bed 101, and a second vibration sensing unit 303 and a second pressure sensing unit 304 are provided at a lower end thereof, and a configuration in which a third vibration sensing unit 305 and a third pressure sensing unit 306 are provided at a top of the third split bed 103. There is shown a configuration in which a fourth vibration sensing unit 307 and a fourth pressure sensing unit 308 are provided on the second split bed 102. The second split bed 102 that supports the head may be provided with sensing units to sense a vibration signal according to a cerebral blood flow or a vibration signal sensed from the face.

The sleep improvement device with a folded mattress of FIG. 7 or 8 is only an example, but through this detailed description, it can be seen that multiple vibration sensing units and pressure sensing units can be installed on each split bed as in FIG. 7 or 8.

In the above, an unrestrained sleep improvement device according to the present disclosure and a sleep improvement method using the same have been examined. Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

In particular, configurations that implement the technical features of the present disclosure included in the block diagrams and flowcharts shown in the drawings attached to this specification represent logical boundaries between the configurations. However, according to an embodiment of software or hardware, the shown configurations and functions thereof are executed in the form of stand-alone software modules, monolithic software structures, codes, services, and combinations thereof, and the functions may be implemented by being stored in a medium executable on a computer provided with a processor capable of executing the stored program codes, instructions, and the like, and therefore, all of these embodiments should also be regarded as falling within the scope of the present disclosure.

Accordingly, the accompanying drawings and technologies thereof describe the technical characteristics of the present disclosure, but should not be simply inferred unless a specific array of software for implementing such technical characteristics is clearly described otherwise. That is, the aforementioned various embodiments may be present, and may be partially modified while having the same technical features as those of the present disclosure, and thus such modified embodiments should also be regarded as falling within the scope of the present disclosure.

Furthermore, the flowchart describes operations in the drawing in a specific sequence, but has been shown to obtain the most preferred result, and it should not be understood that such operations must be carried out in the specific sequence or sequential sequence shown, or that all shown operations must be carried out. In a specific case, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

### DESCRIPTION OF SYMBOLS

10 Layer 20 Vibration unit 201 Actuator 202 Frame 203 Cone surface 205 Edge surface

207 Cap 250 Vibration transmitting body 30 Sensing unit 301 Vibration sensing unit 302 Pressure sensing unit 40 Calculation unit

51 Vibration plate 101, 102, 103 Split bed 21, 22, 23 Vibration unit 301, 303, 305, 307 Vibration sensing unit 302, 304, 306, 308 Pressure sensing unit

1. A sleep improvement device, the device comprising:
a plurality of layers constituting a mattress; and
a vibration unit provided in any one of the plurality of layers to output a vibration signal,
wherein the vibration unit is provided so as to output a vibration signal toward an upper side of the mattress.

2. The device of claim 1, further comprising:
a vibration sensing unit provided in any layer to acquire a vibration signal from the sleep improvement device or a user; and
a pressure sensing unit provided in an arbitrary layer to acquire a pressure signal according to the user's movement.

3. The device of claim 2, wherein the vibration unit comprises an actuator that generates vibration,
wherein the actuator is in contact with a lower surface of a vibration plate, and
wherein the vibration plate is provided to be in contact with a lower surface of the mattress.

4. The device of claim 3, further comprising:
a vibration transmitting body for amplifying or attenuating the vibration signal, the vibration transmitting body that is present between the vibration unit and an upper surface of the mattress.

5. A sleep improvement device, the device comprising:
at least two split beds,
wherein each split bed comprises:
a plurality of layers;
a vibration sensing unit provided in any layer among the plurality of layers to acquire a vibration signal from the split bed or a user's body in contact with an upper surface of the split bed; and
a pressure sensing unit provided in an arbitrary layer to acquire a pressure signal according to the user's movement.

6. The device of claim 5, wherein at least one of the plurality of split beds further comprises a vibration unit that outputs a vibration signal.

7. The device of claim 6, wherein the sleep improvement device comprises:
a first split bed in contact with the user's back or stomach; and
a second split bed in contact with the user's legs.

8. The device of claim 7, wherein the first split bed comprises:
a first-first vibration sensing unit and a first-first pressure sensing unit provided at a top of the first split bed; and
a first-second vibration sensing unit and a first-second pressure sensing unit provided at a bottom of the first split bed.

9. A sleep improvement method using a sleep improvement device,
wherein the sleep improvement device comprises:
a plurality of layers constituting a mattress; and a vibration unit provided in any one of the plurality of layers to output a vibration signal, the vibration unit being provided so as to output a vibration signal toward an upper surface of the mattress, and
wherein the sleep improvement method comprises:
sensing a user's lying posture on a layer of the sleep improvement device; and
controlling the vibration unit to output a vibration signal toward an upper surface of the mattress.

10. A sleep improvement method using a sleep improvement device,
wherein the sleep improvement device comprises at least two split beds,
wherein each split bed comprises: a plurality of layers constituting each split bed; a vibration sensing unit provided in any layer among the plurality of layers to acquire a vibration signal from the split bed or a user's body in contact with an upper surface of the split bed; a pressure sensing unit provided in an arbitrary layer to acquire a pressure signal according to the user's movement; and a vibration unit included in at least one of the plurality of split beds to output a vibration signal, and
wherein the sleep improvement method comprises:
sensing the presence of a user applying pressure to any one of the split beds of the sleep improvement device; and
controlling the vibration unit to output a vibration signal toward an area to which pressure is applied by the user.
